(19) United States
(12) Patent Application Publication
(10) Pub. No.: US 2018/0246122 A1
(43) Pub. Date: Aug. 30, 2018

(54) USE OF ODAM AS PARADENTAL DISEASE BIOMARKER

(71) Applicant: SEOUL NATIONAL UNIVERSITY R&DB FOUNDATION, Seoul (KR)

(72) Inventors: Joo-Cheol PARK, Seoul (KR); Hye-Kyung LEE, Seoul (KR)

(73) Assignee: SEOUL NATIONAL UNIVERSITY R&DB FOUNDATION, Seoul (KR)

(21) Appl. No.: 15/528,360

(22) PCT Filed: Nov. 19, 2015
(86) PCT No.: PCT/KR2015/012456

§ 371 Date: May 19, 2017

(30) Foreign Application Priority Data
Nov. 20, 2014 (KR) 10-2014-0162287

(51) Int. Cl. G01N 33/68; G01N 33/53

(52) U.S. Cl. CPC G01N 33/6893; G01N 2800/18; G01N 2333/47; G01N 33/5308

(57) ABSTRACT

Disclosed is a composition including an ODAM detecting reagent for diagnosis of periodontal diseases, and a kit and a method therefor. A marker according to the present subject matter can conveniently diagnose periodontal disease at an early stage in a noninvasive manner, thereby able to decide effective treatment of periodontal diseases as well as reduction in the ever-increasing medical cost for treating periodontal diseases.

### CROSS-REFERENCE TO RELATED APPLICATIONS

The present application is a national stage application of International Patent Application No. PCT/KR2015/012456, filed Nov. 19, 2015, and claims the benefit of Korean Patent Application No. 10-2014-0162287, filed Nov. 20, 2014 in the Korean Intellectual Property Office, the disclosure of which are incorporated herein.

### STATEMENT OF GOVERNMENT SUPPORT

The invention was made with government support under grant number NRF-2013M3A9B2076480 “Study for commercialization of oral protein derivative” awarded by the National Research Foundation, Republic of Korea.

### BACKGROUND OF THE INVENTION

### Field of the Invention

The present invention relates to biomarkers to diagnose periodontal disease.

### Description of the Related Art

Periodontal disease is a pathological inflammatory condition in the gums and the periodontal tissues surrounding the teeth and caused by bacterial infection or plaque build-up in the teeth. It is called a silent disease due to its slow progression, but which if left untreated may result in tooth loss.

According to the Korea national survey of oral health status in 2012, the morbidity rate of dental caries is decreasing in contrast to periodontal disease, which is increasing.

Also according to the national health insurance service, gingivitis and periodontal disease ranked second only next to acute bronchitis in ambulatory care in 2013.

However, the progression of periodontal disease into advanced stage can be prevented through early detection and treatment. Also the damaged periodontal tissues surrounding the teeth can be recovered through tissue regeneration therapy.

Currently the diagnosis of periodontal disease relies on the subjective symptom of the patients, the determination of periodontal pocket depth using probes in combination with radiography, the determination of the severity of bacterial infection, or the progression status of the periodontal disease determined by gingival recession.

However, for effective treatment of the disease, there are needs to develop indicators or markers enabling a convenient as well as early detection or diagnosis of periodontal disease.

US Patent application publication No. 2008-0027146 discloses biomarkers for diagnosis periodontal disease, which including V-FOS FBJ murine osteosarcoma viral oncogene homolog B matrix metalloproteinase 1, matrix metalloproteinase 3, and caspase 10 and the like.

US Patent application publication No. 2002-0012944 discloses a use of PAI-2 and T-PA as diagnostic markers of periodontal disease.

However, there still exist needs for further development of markers for more efficient and accurate diagnosis of periodontal disease.

### SUMMARY OF THE INVENTION

The present disclosure is to provide a biomarker, which can diagnose or detect the various stages of periodontal disease in a non-invasive way using a sample collected from periodontal pocket.

In one aspect of the present disclosure, there is provided a composition for diagnosis of or detecting the various stages of periodontal disease periodontal disease in a sample comprising an agent for detecting ODAM biomarker. In one embodiment, the sample includes a gingival crevicular fluid or saliva.

In one embodiment of the present disclosure, the biomarker can be advantageously used to diagnose of periodontal disease such as gingivitis, periodontitis or peri-implantitis.

In one embodiment, the agents for detecting ODAM includes reagents for detecting market at the protein level, such as for example a western blot, ELISA, radioimmunoassay, immunodiffusion assay, immunoelectrophoresis, immunohistochemistry, immunopercipitation assay, complement fixation assay, FACS, Mass spectrometry, or protein microarray without being limited thereto. In further embodiment, such regents include for example a monoclonal antibody, a polyclonal antibody, a substrate, an aptamer, an avimer, a peptidomimetics, a receptor, a ligand or a cofactor without being limited thereto.

In other aspect of the present disclosure, there is provided a kit for diagnosis of or detecting periodontal disease at various stages in a sample comprising an agent for detecting ODAM biomarker.

In one embodiment, the kit according to the present disclosure may be provided as ELISA, a dip stick rapid kit, a microarray analysis, a nucleic acid amplification, or a immunoassay.

In still other aspect of the present disclosure, there is provided a method for diagnosis or detecting periodontal disease such as periodontitis or peri-implantitis, comprising providing a biological sample; detecting ODAM at a protein level by contacting the sample with an agent that specifically recognizes ODAM such as antibody from a subject; and correlating the detection result or the concentration with the diagnosis or prognosis of periodontitis or peri-implantitis. In one embodiment the correlation step includes comparing the detection result with a threshold value of the corresponding marker determined in a comparative sample.

In one embodiment, a gingival crevicular fluid or saliva can be advantageously used as a sample for determination of ODAM presence and/or concentration.

In one embodiment, the method may be performed by methods known in the art such as assays including a microarray, an amplification, an antigen-antibody reaction, or a mass spectrometry.

In one embodiment, the subject's non-biomarker information may include a depth of paradental cyst, bleeding, or X-ray test.

### Advantageous Effects

The present compositions, methods and kits based on the use of ODAM as a biomarker can be advantageously used for early diagnosis of periodontal disease, leading to an effective treatment of the periodontal disease and reduction in the medical cost.

### BRIEF DESCRIPTION OF THE DRAWINGS

FIGS. 1A to 1E are results of immunohistochemistry showing the location and time of the expression of ODAM and RhoA in the teeth and gums.

FIGS. 2A to 2E are results of immunohistochemistry showing that the expression of ODAM is decreased in the gum cell affected with inflammation and detected in gingival crevicular fluid.

FIGS. 3A to 3C are graphs showing the depth dependent concentration of ODAM in the periodontal pocket of patients affected with periodontal disease in which the concentration of ODAM was measured by ELISA and expressed as ng/ml in the gingival crevicular fluids collected from the periodontal pocket of the patients. The patients were also examined and classified according to the pocket depth determined by probing (correlation coefficient t=0.167*, Sig.(2-tailed)=0.0127)(FIG. 3A), Clinical attachment level (CAL) of teeth—periodontal cells (correlation coefficient=0.226**, Sig.(2-tailed)=0.0007)(FIG. 3B), and bleeding in the pocket (FIG. 3C). *: Confidence 0.05 (2-tailed); **: Confidence 0.01 (2-tailed).

FIGS. 4A and 4B are results of ODAM concentration determined in the sample obtained from patient affected with peri-implantitis after dental implant surgery.

FIG. 5 is a ELISA result showing that ODAM is detected in saliva of the patients affected with periodontal diseases.

### DETAILED DESCRIPTION OF THE EMBODIMENTS

The present inventions disclosed herein are based, in part, on the discovery that ODAM protein is present in paradental cyst of periodontitis patients. In particular, it has been discovered that ODMA, which is usually highly expressed in junctional epithelial cells during the teeth development and buccal epithelial cells attached to the teeth after surgical periodontal treatment, is no longer expressed in the damaged cells or tissues affected with gingivitis.

It is therefore an aspect of the present invention to provide a use of ODAM as a biomarker for diagnosis or prognosis of gingivitis, periodontitis or peri-implantitis.

A further aspect of the present invention provides a composition for diagnosis or prognosis of gingivitis, periodontitis or peri-implantitis in a sample in need thereof comprising an agent for detecting ODAM biomarker in the sample.

ODAM (ODontogenic AMeloblast-associated protein) is a protein found to be expressed in various cancers such secretory gland cancer and oral cancer and cervical cancer and the like as well as in cells such odontoblasts and ameloblasts. ODAM can increase the cell attachment in cancer cells and thus can prevent or decrease the cancer transition or metastasis. However there are no previous reports that ODAM's function as a biomarker in periodontitis

It has been found in the present disclosure that ODAM is expressed in actin rings of the cells such as macrophages to induce cell attachment, the expression of which is reduced in a pathological lesion due to, for example, an infection. However, it has been found in the present disclosure that ODAM is present in gingival crevicular fluid from patients with inflammatory periodontal disease, the amount of which is increased as periodontal pocket depth is increased.

Currently, a periodontal diseases is usually treated at an advanced stage when the pain is present or the lesion is visible. However, a treatment at an early stage is imperative for an effective treatment since the periodontal pocket depth deepens and the gum are receding as the disease progresses resulting in the tooth damage as well as damages and loss of the bone tissues surrounding the teeth. Thus, the development of biomarkers able to identify the periodontal disease at an early stage can be advantageously used for various purposes such as for the preservation of height and structural integrity of the gums, for maintaining the teeth in a good condition and for preventing the loss of the bone tissues around the teeth. It has been found in the present disclosure that ODAM proteins are released into the gingival sulcus as the attachment between the teeth and the junctional epithelia tissues is lost. Thus in accordance with the present invention, the diagnosis of periodontitis at an early stage is possible by detecting ODAM proteins released into the gingival sulcus. The loss of attachment between the teeth and the junctional epithelia further indicates that the supporting connective tissues underneath the epithelial tissues are also damaged, suggesting an early stage periodontitis. Without being bound by the theory, this may be explained as follows: as the periodontitis progresses, the damages to the tissues including the bone result in deepening the periodontal pocket as the epithelial tissues recedes; however the epithelial tissues still express ODAM proteins in an physiological effort to recover the attachment although it cannot attach themselves to the teeth, which are eventually released into the gingival sulcus. Thus concentration of ODAM released can be correlated with the progression of the periodontal disease. Thus, ODAM can be advantageously used as a convenient and effective marker for diagnosis of periodontal disease particularly at an early stage.

The term periodontal disease or disorder refers to a condition in which the gingival recession and/or inflammation in the surrounding connective tissue and/or the bone are observed, and includes but do not limited to gingivitis, periodontitis and peri-implantitis.

The term peri-implantitis in the present disclosure refers to a destructive inflammatory process affecting the soft and hard tissues including the bone surrounding dental implants, which is developed after the dental implant surgery.

In accordance with the present disclosure, the concentration of ODAM is dependent on the depth of the gingival sulcus and is increasing as the depth increases. The term periodontal pocket refers to a gingival sulcus with a depth of over 3 mm and periodontal pocket and gingival sulcus may be interchangeably used and the exact meaning can be determined within the context. Thus the marker ODAM of the present disclosure can be advantageously used for diagnosing periodontal diseases including gingivitis, periodontitis and peri-implantitis at various stages of development as they progress. For example periodontitis may be divided into three stages of early, moderate and advanced. The early stage of periodontitis is characterized by at least one of pathological condition including bleeding at probing, periodontal pocket with 3 to 4 mm in depth, local gingival recession, the loss of attachment (3-4 mm), the bone loss and class I furcation lesion. The moderate stage of periodontitis is characterized by at least one of pathological condition including bleeding at probing, periodontal pocket with 4 to 6 mm in depth, the loss of attachment (4-6 mm), the bone loss (horizontal and/or vertical), class I or II furcation lesion and class I loose teeth or crown to root ratio of 1:1. The advanced stage is characterized by at least one of pathological condition including bleeding at probing, periodontal pocket with depth over 6 mm, the loss of attachment (over 6 mm), class II and/or III furcation lesion, class II and/or III loose teeth, the bone loss (horizontal and/or vertical), and crown to root ratio of at least 2:1.

The periodontal disease or disorder of the present disclosure also includes its subclasses such as adult periodontitis (for example, periodontitis associated with plaque), early-onset periodontitis (for example, pediatric periodontitis, rapidly progressive periodontitis), periodontitis associated with systematic disease, ulcerative gingivitis, or peri-implantitis.

Gingivitis shows symptoms such as inflammation of the gums, gum bleeding, or pseudo-pockets. The gingivitis of the present disclosure also includes but is not limited to, gingivitis associated with plaque, chronic gingivitis, acute gingivitis, necrotizing ulcerative gingivitis, gingivitis associated with systematic disease or drug related gingivitis (for example, inflammation of the gum due to hormone, gingivitis due to drugs, or gingival erythema and the like), or gingivitis due to infection.

It was discovered in the present disclosure that ODAM protein is expressed in healthy gums, but is not present in the tissues affected with inflammation but is present in gingival crevicular fluid. Without being bound by the theory, this is due to that ODAM proteins present in the cells are released into the gingival sulcus as the epithelial cells detaches themselves from the teeth as the cells are damaged.

The term diagnosis as used herein refers to determining the disease or disorder susceptibility of a subject, determining whether a subject has a specific disease or disorder, determining the prognosis (for example, identification of transitional status, stages or progression of disease or determining the response to treatments) of a subject who has specific disease or disorder, or therametrics (for example, monitoring the status of a subject to provide the information on the efficacy of treatment).

The term biomarker or diagnosis marker as used herein refers to an agent that may discriminate affected tissues or cells from normal cells or a properly treated tissues or cells, and comprises a biological molecule and the like, such as proteins or nucleic acid molecules, the level of which is changed or increased or decreased in affected tissues or cells compared with normal control samples.

The biological sample or material of the present disclosure is a substance or a mixture of the substances that contain or are expected to contain/express one or more of the present biomarkers, and includes cells, tissues or bodily fluids from an organism, particularly human. Also the sample includes cells or tissues cultured in vitro as well as those derived directly from an organism. Also the fractions or derivatives of the cells or tissues are included. When cells or tissues are used, lysates thereof may also be used. In one embodiment of the present disclosure, particularly saliva or gingival crevicular fluid from gingival sulcus can be used advantageously for the present purpose.

The term detection or detecting as used herein refers to a qualitative or quantitative determination and/or determination of changes in patterns and/or profiles of the expression. The detection includes a determination of the presence and/or absence as well as the levels of the present markers. The present markers may be detected using the methods known in the art, and the person skilled in the art would be easily able to select appropriate methods for the detection.

In the present disclosure, the term detecting agents refer to materials that are able to specifically recognize ODAM at the protein or nucleic acid level.

The agents that can detect the present marker at a protein level include for example materials used for Western blot, ELISA (Enzyme linked immunoabsorbent assay) a radioimmunoassay, an immunodiffusion, an immunoelectrophoresis, an immunostaining, an immunoprecipitation, a complement fixation assay, or a protein array such as antigen array and the like. Or a system based on labeled beads, a binding with a labeled antibody in solution/suspension and a detection by flow cytometry, or a mass spectrometry, and the like. These methods are known and documents such as chip-based capillary electrophoresis: Colyer et al. 1997. J Chromatogr A. 781(1-2):271-6; mass spectroscopy: Petricoin et al. 2002. Lancet 359: 572-77; eTag systems: Chan-Hui et al. 2004. Clinical Immunology 111:162-174; microparticle-enhanced nephelometric immunoassay: Montagne et al. 1992. Eur J Clin Chem Clin Biochem. 30:217-22 may be referred.

In one embodiment, an immunoassay using sandwich system like ELISA (Enzyme Linked Immuno Sorbent Assay), or RIA (Radio Immuno Assay) and the like may be used for quantitative and/or qualitative detection of the present markers. In this system, the biological samples are reacted with a first antibody fixed to a solid substrate/support such as a glass, a plastic (for example, polystyrene), polysaccharides, a bead, a nylon or nitrocellulose membrane or a microplate well to form a complex and the complex is then allowed to react with an second antibody that is usually labeled with agents that can be detected directly or indirectly such as radioactive substances like ³H or ¹²⁵I, fluorescent materials, chemiluminescent substances, hapten, biotin, or digoxygenin and the like. In some cases, the labeling materials are conjugated with an enzyme such as horseradish peroxidase, alkaline phosphatase, or maleate dehydrogenase that is able to produce colors or color changes or illuminate in the presence of appropriate substrates.

Other methods based on immune reaction may also be used. In one embodiment, an Immuno Electrophoresis such as an Ouchterlony plate, a Western blot, a Crossed IE, a Rocket IE, a Fused Rocket IE, or an Affinity IE, which can detect the markers simply by antigen-antibody reaction may be used.

The immunoassay or immunostaining methods as described above are for example disclosed in the following literatures: Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, N J, 1984 etc. The intensities of the signals generated by the immunoassay mentioned above are then analyzed, namely compared with the signals from appropriate controls for diagnosis of the disease and the like.

The agents which may be used for the methods described above include but are not limited to, a monoclonal antibody, a polyclonal antibody, a substrate, an aptamer, an avimer, a peptidomimetic, a receptor, a ligand and a cofactor.

In one embodiment, the agents for detecting the present marker are an antibody specifically recognizing the present marker, and are able to quantitative and/or qualitative analysis of the present marker in the biological sample of interest according to the present disclosure. The antibody may be provided fixed on a solid substrate/support such as a glass, a plastic (for example, polystyrene), polysaccharides, a bead, a nylon or nitrocellulose membrane or a microplate well.

In other aspect, the detecting agents which may be used for the present disclosure may be provided as a type of array such as microarray or chip, and the references for the array preparation technology may be found in for example, Schena et al., 1996, Proc Natl Acad Sci USA. 93(20):10614-9; Schena et al., 1995, Science 270(5235):467-70; and U.S. Pat. Nos. 5,599,695, 5,556,752 and 5,631,734. The detecting agents that may be provided as attached to an array substrate include, but are not limited to, antibodies specifically recognizing the present marker, or antibody fragments, or aptamers, avimers (avidity multimer) or peptidomimetics.

The detecting agents may also be labeled for the direct or indirect signal detection in sandwich forms. In the case of direct labeling method, biological samples such as serum to be used for array may be labeled with a fluorescent material such as Cy3 and Cy5. In the case of indirect labeling, unlabeled samples are allowed to bind to the detecting agent which is attached to the array, and then the target or marker proteins are detected by the labeled antibodies which specifically recognize the marker or target. In the case of sandwich methods, the sensitivity and specificity of the detection is usually high and able to detect the marker at the pg/mL level. In addition, labeling agents including such as radioactive materials, agents that produce visible colors under proper condition, magnetic particles and high-density electron particle and the like may also be used. The signals may be analyzed using device such as scanning confocal microscopes, which may purchased from Affymetrix, Inc. or Agilent Technologies, Inc and the like.

In other aspect, the present disclosure relates to kits for detecting or diagnosing gingivitis, periodontitis or peri-implantitis comprising agents or means to detect ODAM.

In one embodiment, the present kits may be provided as an ELISA kit, a dip stick rapid kit, gene amplification or immune analysis kit. The agents which may be included in the kits can be appropriately selected by the ordinary person in the art.

In one embodiment, an ELISA or a dip stick rapid kit is used, and in this case, the antibodies to detect the present markers may be provided as conjugated to a substrate/support, for example to wells of a multi-well plate or surface of a glass slide or a nitrocellulose paper. A dip stick is a form widely used in POCT (point of care technology) in which the biomarkers of the present disclosure may be detected by employing one or more antibodies that are conjugated to a substrate such as nitrocellulose paper which is then contacted with a sample such as serum for example by dipping one end of the stick to the serum and then the sample is moved through the substrate by a capillary action, and the markers are detected by a color change that is developed when the markers of interest are bound by the antibody attached to the substrate.

The kits or compositions of the present disclosure may also include one or more additional components as needed for the detection of the present marker(s), which include, for example, binding buffers, reagents for preparing biological samples, syringes for collecting biological samples or negative and/or positive controls. Also, the kits of the present disclosure may also include instructions for using the kit to detect the markers according to the present disclosure.

In other aspect, the present disclosure relates to a method for detecting a biomarker to provide information as to diagnose or prognosis of periodontal disease. In one embodiment, the method comprises a step of detecting ODAM at a protein level in a sample from a subject; and a step of correlating the detection result with diagnosis or prognosis of periodontitis or peri-implantitis.

In the present methods, the changes of the present biomarker at the protein level in the sample from a subject in need of diagnosis compared to the result from control sample indicate the presence of periodontal disease or peri-implantitis, which can be correlated to the diagnosis or prognosis of periodontal disease or peri-implantitis.

In one embodiment of the present methods, the correlation step includes comparing the detection results with a threshold value of the corresponding marker determined in a comparative sample. The comparative samples include samples from a healthy or normal subject or samples from a subjected treated or recovered from the diseases of interest after undergoing proper therapy.

For example, the cutoff ranges for the present biomarker may be determined based on the results obtained from comparative samples. When the subject suspected of the disease show an increase of at least about 50% in the value determined for a marker, the subject may be diagnosed to have a periodontitis or peri-implantitis. In particular, when the value determined in a subject suspected of the disease has increased over about 2 times, for example, compared to the appropriate cutoff or threshold, the subject may be diagnosed as an advanced stage of periodontitis, enabling the early diagnosis of the disease.

Also, the level of the present biomarker may be determined to confirm that the level is recovered to a normal level in a sample from a patient undergone appropriate treatment, which enables the determination of the efficacy of the therapy and the follow-up of the treatment. The present biomarker may be used alone or in combination with other methods known in the art to diagnose periodontal disease or peri-implantitis.

The biological samples employed for the present methods, the methods to detect the biomarkers and reagents therefor are described as hereinbefore. The present methods are particularly suitable for a mammal, particularly human beings. The human subject includes a subject suspected of periodontal disease or peri-implantitis, or others who are not suspected of periodontal disease or peri-implantitis, but needs diagnosis thereof.

The present methods may be used in combination with other clinical information determined using methods known in the art such as the depth of periodontal pocket, bleeding and/or X-ray readings and the like without being limited thereto.

The present disclosure is further explained in more detail with reference to the following examples. These examples, however, should not be interpreted as limiting the scope of the present invention in any manner.

### EXAMPLES

Reagent and Antibodies:

fibronectin and laminin were purchased from Sigma-Aldrich and collagen from Roche. Antibodies to ODAM were prepared in Rabbit using peptide from amino acid residue 102-114 and 241-251. RhoA, ROCK, p-myosin, p-paxillin, paxillin, E-cadherin antibodies were purchased from Cell Signaling, and active RhoA (GTP-RhoA) antibody from Biosource, and ARHGEF5, Integrin av, Integrin □1, Integrin b6, cytokeratin, HA, E-cadherin antibodies from Santa Cruz Biotechnology, and Flag antibody from Sigma-Aldrich, and F-actin antibody from Invitrogen.

Tissue Preparation and Immunohistochemistry:

All experiments involving animals were performed according to the protocols (SNU-111013-2) approved by IACUC (Institutional Animal Care and Use Committee) of Seoul National University. Rat teeth of 16 days, 20 days (P20), 26 days (P26) of birth and Mice teeth of 10 day (P10) of birth were decalcified in 10% EDTA (pH 7.4), embedded in paraffin, and processed for immunohistochemistry. The human tooth with inflammatory periodontitis extracted for implant was provided by Dr. Sang Joun Yu (Chosun University School of Dentistry, Gwangju, Korea). Sections were incubated overnight with a primary antibody of anti-ODAM, RhoA, active-RhoA or F-actin. As a secondary antibody biotinylated goat anti-rabbit IgG (1:200, Vector Labs Burlingame, Calif.) was used with ABC kit (Vector Labs) to develop.

Cell Culture and Differentiation:

two types cell lines used in the experiment were cultured in a 5% CO₂ atmosphere at 37° C. Mouse ameloblast, immortalized ameloblast-lineage cells (ALCs) (ALC, provided by Dr. T. Sugiyama, Akita Medical School, Japan) were cultured in minimum essential medium (MEM) supplemented with 5% heat inactivated fetal bovine serum (FBS), 10 ng/ml of the recombinant human epithelial growth factor (EGF; Sigma-Aldrich), and antibiotic-antimycotic (Invitrogen) on collagen-coated dishes in a 5% CO₂ atmosphere at 37° C. HAT7 (apical bud cells) cells (from Dr. Harada H, Department of Oral Anatomy II, Iwate Medical College School of Dentistry, Morioka, Japan), were grown and maintained in DMEM/F12 (Gibco BRL) supplemented with 10% FBS and antibiotics in a 5% CO₂ atmosphere at 37° C. RAW264.7 (osteoclast) cells, which are macrophage-like cell line derived from Balb/c mice, were grown and maintained in DMEM supplemented with 10% FBS and antibiotics in a 5% CO2 atmosphere at 37° C. ALC cells and HAT cells were seeded on slides coated with collagen, fibronectin, or laminin as appropriate. Macrophages from bone marrow were grown in a-MEM supplemented with 10% heat inactivated FBS. To induce differentiation, 80%-90% confluent cells were cultured in MEM supplemented with 5% FBS, ascorbic acid (50 μg/ml), and β-glycerophosphate (10 mM) for up to 2 weeks.

Western Blot:

To prepare whole cell extracts, cells were washed three times with PBS, scraped into 1.5 ml tubes, and pelleted by centrifugation at 12,000 rpm for 2 min at 4° C. After removal of the supernatant, pellets were suspended in lysis buffer [50 mM Tris-Cl (pH 7.4), 150 mM NaCl, 1% NP-40, 2 mM EDTA (pH 7.4)] and incubated for 15 min on ice. Cell debris was removed by centrifugation. Proteins (30 μg) were separated by 10% SDS-PAGE and transferred to nitrocellulose membranes (Schleicher & Schuell BioScience, Dassel, Germany). Membranes were blocked for 1 h with 5% nonfat dry milk in PBS containing 0.1% Tween 20 (PBS-T), and incubated overnight at 4° C. with the primary antibody diluted in PBS-T buffer (1:1000). After washing, membranes were incubated for 1 h with secondary IgG antibodies anti-mouse (sc-2031), anti-rabbit (sc-2004), or anti-goat (sc-2768) (1:5000 dilution) conjugated with horseradish peroxidase (all from Santa Cruze Biotechnology). Labeled protein bands were detected using an enhanced chemiluminescence system (GE Healthcare, UK). The bands developed on a film were then quantified using image analyzer.

Gene Expression Profiling:

Publicly available gene expression datasets were downloaded from gene expression omnibus (GEO) (accession number, GSE4250 to Hereditary gingival fibromatosis, GSE2255 to Integrin beta-6 deficiency model of emphysema, and GSE9723 to Gingival epithelial cell line response to oral pathogen infections).

Study Subjects and Clinical Examinations:

This study protocol was approved by the Institutional Review Board for Human Subjects of the Korea University Anam Hospital (IRB No. ED13162) and Seoul National University Hospital at Bundang (IRB No. B-1410-271-003). Four unrelated, systemically healthy adults (three men and one woman) were included in the study and 10 subjects with periodontal disease were included and assessed for the expression of ODAM in relation to the presence or absence of inflammation. Also from four subjects with periodontal disease, samples were obtained from at least 4 sites of the teeth of one quadrant on one jaw which is containing the teeth showing the deepest probing depth and the contralateral quadrant of the opposite jaw, so total 222 samples were collected from at least 10 teeth of each subject. Periodontal examination included the assessment of plaque score, probing pocket depth, loss of attachment and bleeding on probing. Probing pocket depth was performed at 4 sites/teeth. All subjects were diagnosed with chronic periodontitis. Before sample collection from teeth, supragingival plaque was removed with a hand curette. Each tooth site was gently dried for 10 seconds with compressed air and isolated from saliva with a cotton roll. Samples were obtained from 4 sites of one tooth using absorbing paper strips (Oraflow, Smithtown, N.Y., USA). Paper strips were pushed toward the sulcus until a slight resistance was felt; they remained in place for 30 seconds at the mesial and distal surface of each tooth. Paper strips were placed in a single, labeled tube containing 100 μl phosphate-buffered saline and the tubes were then transported to the laboratory and stored at −70° C. until ELISA analysis.

ELISA:

The concentration of ODAM in gingival sulcus fluid were determined using ODAM ELISA kit (CUSABIO company) according to the manufacturer's instruction. Statistics were performed using Student's T-test and Analyses was performed using SPSS (SPSS, version 19, SPSS, Chicago, Ill. USA).

### Example 1. Analysis of the Expression Pattern of ODAM and RhoA During the Development in Odontoblast and Junctional Epithelium (JE) and Identification of a Biomarker

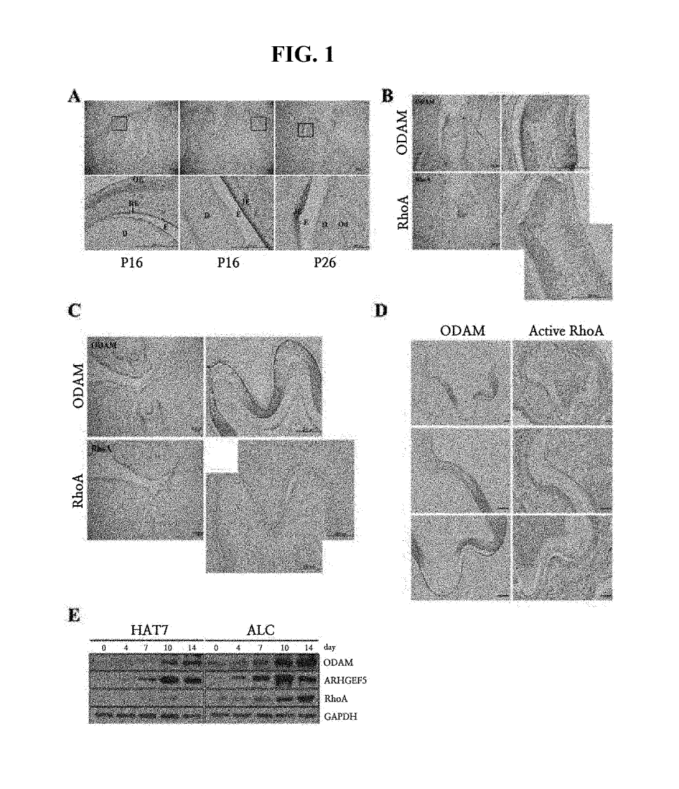

The expression of ODAM at the interface of gum-ameloblast during the tooth development were determined by immunohistochemistry. Results are shown FIG. 1. As shown in FIG. 1, the reducing enamel organ showed immunoreactivity for ODAM, which was restricted to the basal cells of the OE in first molars of postnatal 16 day rats. ODAM was revealed in the supranuclear compartment of reducing ameloblasts and showed a more diffuse labeling at the interface. At 1st molar in postnatal 20 day rats, there was strong immunoreactivity for ODAM, in cell clusters situated between the reducing enamel organ and the OE. Also, ODAM were present at the interface with the tooth. In 26-day-old rats in which all three molars were fully erupted and the JE was well-established, ODAM was found among cells of the JE as a pericellular labeling (FIG. 1A).

Also the expression pattern of ODAM and RhoA protein in the junctional epithelium and the developing molar tooth germs was determined by immunohistochemistry. In postnatal 20 day rats, ODAM expressed strongly in JE. Also, RhoA were localized in same area (FIG. 1B). In postnatal 10 day mouse, ODAM expressed in ameloblasts, enamel matrix, and ameloblast-tooth interface. Interestingly, the expression of ODAM correlated with active RhoA (GTP-RhoA) expression in ameloblast and ameloblast-tooth interface (FIG. 1C, 1D).

Selective and time-dependent induction of ODAM, ARHGEF5, and RhoA was observed during ameloblast differentiation in HAT7 and ALC cells using Western blots. The expression of ODAM gradually increased with time during culture (FIG. 1E). Likewise, ARHGEF5 and RhoA were steadily expressed during ameloblast differentiation (FIG. 1E). These findings suggest that ODAM, ARHGEF5, and RhoA are functionally related to the differentiation and maturation of ameloblasts and the formation of JE. Particularly ODAM was found to be strongly expressed in oral epithelial cells attaching the teeth after surgical periodontal treatment and JE during the teeth development. Thus ODAM was chosen as a biomarker.

### Example 2. Analysis of Localization and Expression Pattern of ODAM in Damaged JE In Vivo

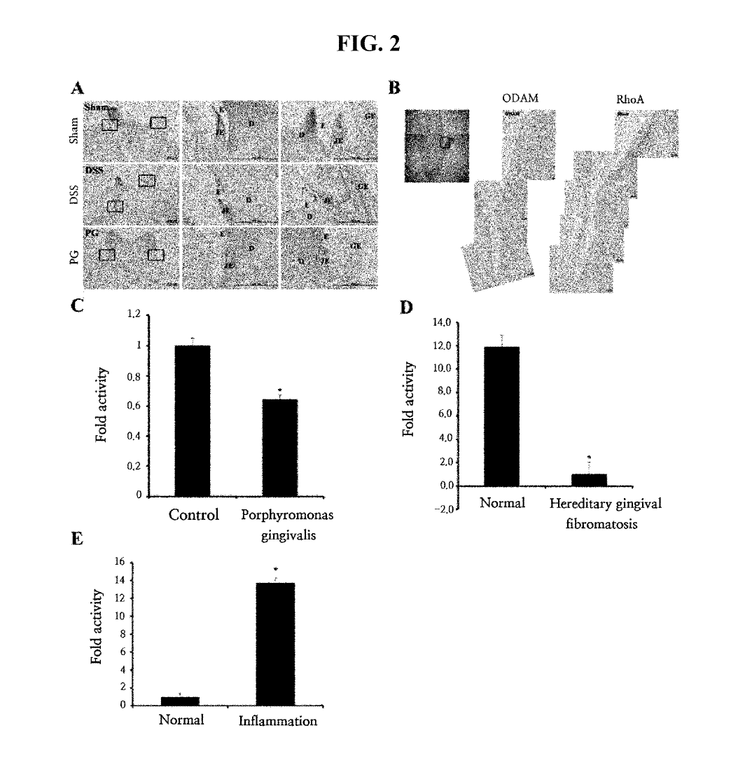

The presence and distribution of ODAM and RhoA in damaged JE following inflammation were determined by immunohistochemistry analyses with damaged JE of mouse and human. As shown in FIG. 2, ODAM was detected in normal JE but not in inflammated JE of mouse by chemical drug (DSS or PG). As shown in FIG. 2, the expression of ODAM was decreased in JE affected with inflammation, and ODAM was increased in gingival crevicular fluid. Interestingly, because the drug ingested through the mouth of mouse, the JE of lingual portion was a severe defect but the JE of labial portion which was less affected by drug showed natural phenotype similar to the normal (FIG. 2A), indicating that expression of ODAM is conserved in normal JE. In periodontal disease, JE transforms to an invasive pocket epithelium, which shows unique pathological features such as ulcerations and formation of epithelial ridges that are surrounded by a heavy inflammatory infiltrate. To investigate the expression of ODAM in the invasive pocket epithelium, the specimens were collected from periodontally diseased tissue from patients, who extracted a tooth with deep periodontal pockets around their teeth for implant. ODAM and RhoA were not detected in damaged JE of human by inflammation compared with normal tissue (FIG. 2B, C).

To confirm the ODAM expression in inflammated JE, we used microarrays data from NCBI GEO data set to detail the ODAM expression in gingival epithelial cells modulated with the oral pathogenic Porphyromonas gingivalis (PG). ODAM protein was inhibited by PG in JE which was infected by PG (FIG. 2D). Hereditary gingival fibromatosis (HGF), known as idiopathic gingival hyperplasia, is a group of benign disorders characterized by enlargement of the gingivae. HGF associated with aggressive periodontitis which typically results in severe, rapid destruction of the tooth supporting apparatus. We analyzed the expression of ODAM using GEO data which was analyzed using gingival tissues from a patient with HGF. ODAM expression decreased in gingival tissues with HGF compared with gingival tissues of normal patients (FIG. 2E). These results suggest that the expression of ODAM, which expressed in healthy JE, is decreasing with the on-set of periodontal disease and is increasing in gingival crevicular fluid, indicating that ODAM can be advantageously used for biomarker for periodontal disease.

### Example 3. Analysis of the Concentration of ODAM in Gingival Cervical Fluids Obtained from Various Depth of Gingival Sulcus from Patient Affected with Periodontal Disease

In periodontal disease, as the disease progresses, the teeth and JE are detached from each other, the gingival sulcus are deepen, and the bone loss are becoming more severe. Thus as the depth of the gingival sulcus from which the fluid is collected is increasing, it indicates that the periodontal disease is more progressive stage of the disease. As described in the method section, the concentration of ODAM was determined along the depth of gingival sulcus.

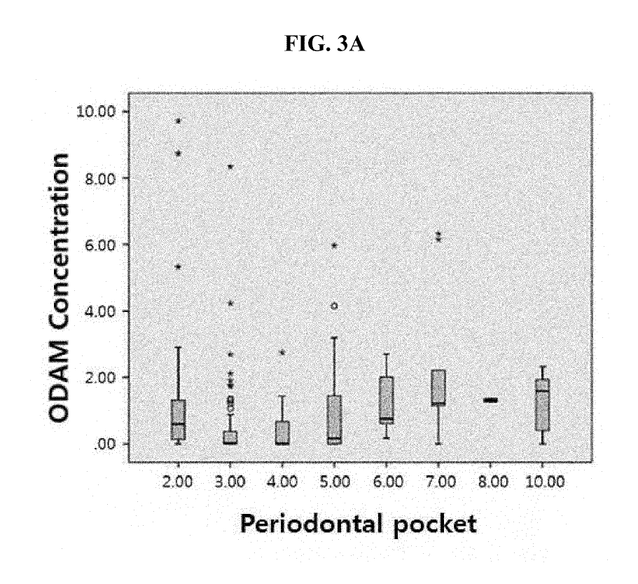

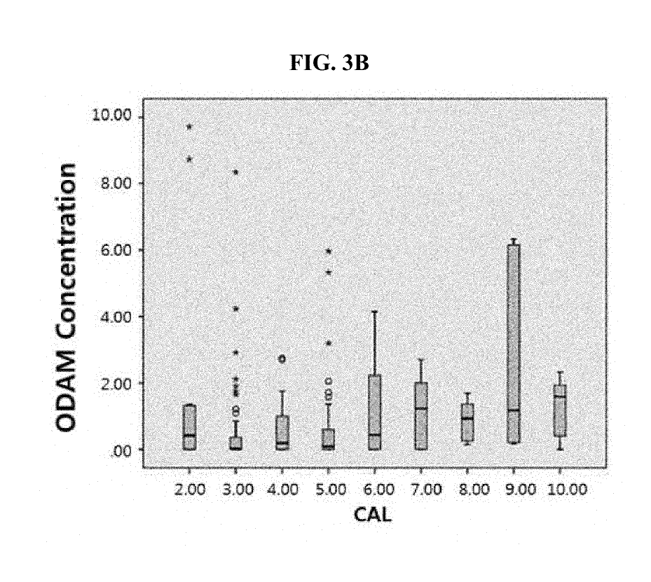

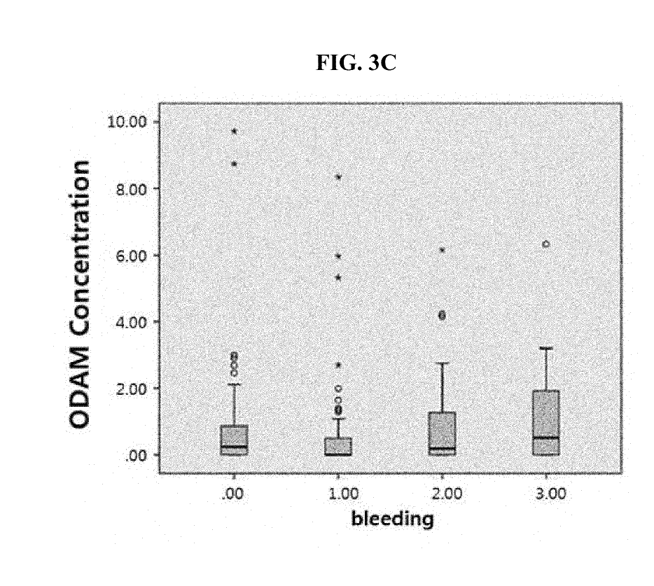

Results are shown in FIGS. 3a and 3b. FIG. 3 is a graph showing the concentration of ODAM along the depth of gingival sulcus from which the samples were obtained, in which probing pocket depth, periodontal pocket (FIG. 3a), the loss of attachment between teeth-periodontal tissue in the pocket (Clinical Attachment Level, CAL) (FIG. 3b) and Bleeding in the pocket (FIG. 3c) were determined. Then the concentration (ng/ml) of ODAM was determined from the gingival sulcus fluids collected therefrom by ELISA. The graph shows the concentration of ODAM along the depth of the pocket. Probing pocket depth was measured/determined as compared to the depth of a normal pocket. The relationship between ODAM and probing pocket depth, that is, the correlation coefficient is 0.167, Sig.(2-tailed)=0.0127 (confidence level 0.05); the correlation coefficient of CAL and ODAM is 0.226, Sig.(2-tailed)=0.0007 (confidence level 0.01). This indicates nearly a confidence level of about 100%. Thus this indicates that ODAM of the present marker can be advantageously used for diagnosing early stage of periodontal disease as well as advanced stage of disease and therebetween.

### Example 4. Analysis of the Concentration of ODAM in Gingival Cervical Fluids from Patient Affected with Peri-Implantitis

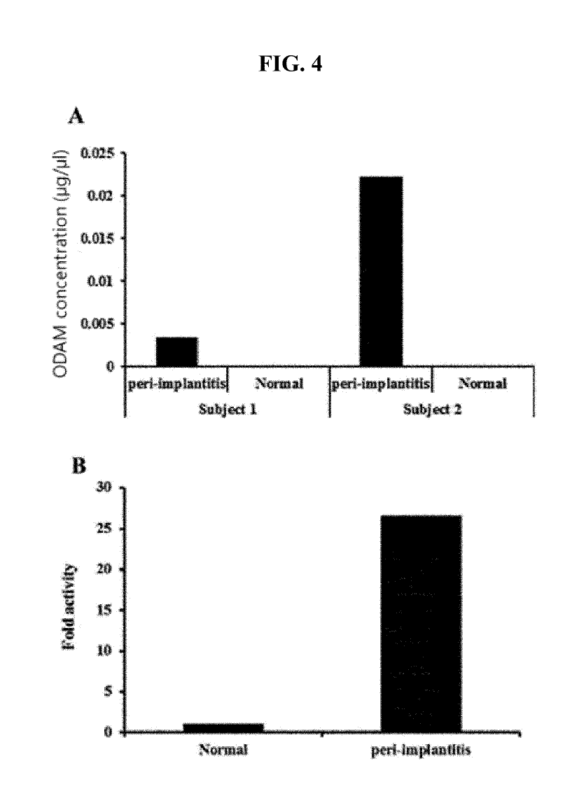

After dental implant surgery, gingival cervical fluids were collected from the site affected with peri-implantitis and the site not affected with peri-implantitis. Then the concentration of ODAM was determined as described in method section. As shown in FIG. 4a, it was found that the concentration of ODAM is increased at the site affected with peri-implantitis compared to non-affected site.

Also FIG. 4b shows that the concentration of ODAM is significantly increased in sample from peri-implantitis patient compared to the sample from normal control periodontal tissue who has not undergone dental implant surgery.

### Example 5. Analysis of the Concentration of ODAM in Saliva from Patient Affected with Periodontal Disease

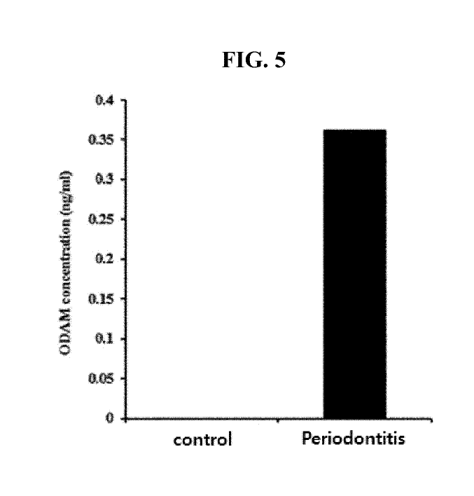

It was confirmed that ODAM proteins are released into gingival cervical fluid as periodontal disease worsens, which was confirmed by ELISA. Thus ODAM can be detected also in saliva, As described in the method section, patients affected with periodontal disease were assessed for severity, and the concentration of ODAM was determined from saliva collected. As shown in FIG. 5, it was found that ODAM was not in saliva from patients without inflammation; however ODAM was present in saliva diagnosed with periodontal disease.

The various singular/plural permutations may be expressly set forth herein for sake of clarity. Although a few embodiments of the present disclosure have been shown and described, it would be appreciated by those skilled in the art that changes may be made in this embodiment without departing from the principles and sprit of the invention, the scope of which is defined in the claims and their equivalents.

Unless defined or interpreted otherwise, all technical and scientific terms and any acronyms used herein have the same meanings as commonly understood by one of ordinary skill in the art in the field of the invention.

1.-14. (canceled)

15. A method of detecting ODAM in a patient, the method comprising:
a. obtaining an oral liquid sample from a patient; and
b. detecting whether ODAM is present in the sample by contacting the sample with an ODAM antibody and detecting binding between ODAM and the antibody.

16. The method of claim 15, wherein the patient is affected with gingivitis, periodontitis or peri-implantitis with damaged JE.

17. The method of claim 15, wherein the oral liquid sample is a gingival crevicular fluid or saliva.

18. A method of diagnosing periodontal disease in a patient, the method comprising:
a. obtaining an oral liquid sample from a patient; and
b. measuring the concentration of ODAM by contacting the sample with an ODAM antibody and detecting binding between ODAM and the antibody; and
c. diagnosing the patient with periodontal disease when the concentration of ODAM is increased compared to the concentration determined in a normal sample.

19. The method of claim 18, wherein the patient is affected with gingivitis, periodontitis or peri-implantitis with damaged JE.

20. The method of claim 18, wherein the ODAM antibody is from a mouse, a goat or a rabbit.

21. A method of diagnosis of gingivitis, periodontitis or peri-implantitis with damaged JE in a subject in need thereof, comprising:
a. obtaining an oral liquid sample from a patient; and
b. measuring an ODAM protein concentration by contacting the sample with an ODAM antibody and detecting binding between ODAM and the antibody; and
c. correlating the patient with gingivitis, periodontitis or peri-implantitis with damaged JE when the concentration is increased compared to the concentration determined in a normal sample.

22. The method of claim 21, wherein the oral liquid sample is a gingival crevicular fluid or saliva.

23. The method of claim 21, wherein the ODAM antibody is from a mouse, a goat or a rabbit.
